(19) United States
(12) Patent Application Publication
(10) Pub. No.: US 2021/0178037 A1
(43) Pub. Date: Jun. 17, 2021

(54) INTRA-VENTRICULAR PULSATILE ASSIST SYSTEM (IV-PAS)

(71) Applicant: Medtronic, Inc., Minneapolis, MN (US)

(72) Inventor: Stefan KRACKER, Sonthofen (DE)

(21) Appl. No.: 17/096,436

(22) Filed: Nov. 12, 2020

Related U.S. Application Data
(60) Provisional application No. 62/948,539, filed on Dec. 16, 2019.
(60) Provisional application No. 62/964,225, filed on Jan. 22, 2020.

(51) Int. Cl. A61M 1/10; A61M 1/12

(52) U.S. Cl. CPC A61M 1/1074; A61M 1/122; A61M 2205/50; A61M 1/1087; A61M 1/1031; A61M 1/1006

(57) ABSTRACT

An intra-ventricular pulsatile assist system is provided including an intra-ventricular blood pump including a chamber having a distal portion and a proximal portion opposite the distal portion, the proximal portion and the distal portion defining an axis extending therebetween and the distal portion defining an outlet; a valve at the distal portion of the chamber, the valve including a closed position in which the outlet is sealed and an open position in which the outlet is unsealed; and a control circuit including a processor in communication with the blood pump, the processor having processing circuitry configured to determine a pressure value in the chamber and transition the valve between the closed position and the open position when the pressure value in the chamber deviates from a predetermined threshold value.

### CROSS-REFERENCE TO RELATED APPLICATION

This application claims the benefit of U.S. Provisional Application Ser. No. 62/948,539, file Dec. 16, 2019 and claims the benefit of U.S. Provisional Application Ser. No. 62/964,225 filed Jan. 22, 2020.

### FIELD

The present technology is generally related to implantable blood pumps.

### BACKGROUND

Implantable blood pumps are commonly used to assist the pumping action of a failing heart and typically include a housing with an inlet, an outlet, and a rotor mounted therein. The inlet may be connected to a chamber of the patient's heart, typically the left ventricle, using an inflow cannula. The outlet may be connected to an artery, such as the aorta. Rotation of the rotor drives blood from the inlet towards the outlet and thus assists blood flow from the chamber of the heart into the artery.

A known type of blood pump is a ventricular assist device (“VAD”) with examples including, but not limited to, the HVAD® pump and the MVAD® pump. VADs having continuous flow (CF) systems deliver constant power with constant rotational speed, rather than the power adjusting relative to a patient's remaining heart activity. As a result of the blood pump performing the pumping action to the exclusion of the heart, there is minimal to no opportunity for a patient's heart to recover, for example, after a myocarditis. VADs having pulsatile flow (PF) system are designed to mimic the pumping action of the heart. As such, pulsatile flow systems typically result in less weaning time for patients able to eventually discontinue use of the blood pump when compared to continuous flow systems.

Further, known VADs transport blood from a patient's ventricular apex through an artificial tube to the ascending or descending aorta (left ventricular assist device (“LVAD”)) or to the pulmonary artery (right ventricular assist device (“RVAD”)). The space consumed by the tube is normally of insufficient size to combine two devices to form a biventricular VAD (“BiVAD”).

### SUMMARY

The techniques of this disclosure generally relate to an intra-ventricular pulsatile assist system and blood pump configured to time an ejection of fluid from the blood pump in accordance with a cardiac cycle of the patient to promote perfusion and the strengthening of the patient's natural heart or provide permanent pulsatile circulatory support.

In one aspect, the present disclosure provides an intra-ventricular pulsatile assist system including an intra-ventricular blood pump having a chamber including a distal portion and a proximal portion opposite the distal portion, the proximal portion and the distal portion defining an axis extending therebetween and the distal portion defining an outlet; a valve at the distal portion of the chamber, the valve including a closed position in which the outlet is sealed and an open position in which the outlet is unsealed; and a control circuit including a processor in communication with the blood pump, the processor having processing circuitry configured to determine a pressure value in the chamber and transition the valve between the closed position and the open position when the pressure value in the chamber deviates from a predetermined threshold value.

In another aspect, the disclosure provides the system including the predetermined threshold value being a target pressure value for the chamber, and the chamber is of an expandable material.

In another aspect, the disclosure provides the system including the predetermined threshold value being a predetermined time interval associated with a cardiac cycle of a patient having the blood pump implanted in the patient.

In another aspect, the disclosure provides the system including the processing circuitry being further configured to execute a plurality of phases, control a pump speed of the blood pump relative to the plurality of phases, and control a pump current of the blood pump relative to the plurality of phases, and wherein the pump speed and the pump current define the pressure value in the chamber.

In another aspect, the disclosure provides the system including the processing circuitry being further configured to increase the pump speed and increase the pump current relative to one of the group consisting of an aortic pressure and a pulmonary pressure of the patient.

In another aspect, the disclosure provides the pump speed and the pump current defining a one-way fluid flow path along the axis of the chamber from the proximal portion of the chamber to the distal portion of the chamber.

In another aspect, the disclosure provides the plurality of phases including a cycle start and a filling phase, and the processing circuitry is further configured to increase the pump speed and increase the pump current relative to the cycle start during the filling phase, and wherein the pump speed and the pump current define a target pressure value in the chamber timed in association with a transition of a cardiac cycle of the patient from a diastole phase to a systole phase.

In another aspect, the disclosure provides the system including a housing coupled to the proximal end of the chamber and including a rotor or any other fluid moving device disposed therein; a fixation device coupled to the housing and having a heartbeat sensor coupled thereto; an intracorporal driveline extending from the housing and being in communication with the rotor; and a power source coupled to the intracorporal driveline.

In one aspect, the present disclosure provides an intra-ventricular pulsatile assist system including an intra-ventricular blood pump having a chamber defining a fluid cavity and an outlet in fluid communication with the fluid cavity; a valve coupled to the chamber and having a closed position in which the outlet is sealed and an open position in which the outlet is unsealed; and a housing couple to the chamber and having a rotor and a motor therein; and a control circuit including a processor in communication with the blood pump, the processor having processing circuitry configured to control a pump speed of the rotor and a pump current of the motor, the pump speed and the pump current defining a pressure value in the chamber; determine the defined pressure value in the chamber; compare the determined pressure value in the chamber to a predetermined threshold value associated with one of a group consisting of an aortic pressure value and a pulmonary pressure value of a patient having the blood pump implanted in the patient; and transition the valve between the closed position and the open position when the determined pressure value deviates from the predetermined threshold value.

In another aspect, the disclosure provides the system including the predetermined threshold value being a target pressure value for the chamber but may be also related to another pressure value (e.g. aortic pressure or pressure at the aortic valve.

In another aspect, the disclosure provides the system including the predetermined threshold value being a predetermined time interval associated with a cardiac cycle of the patient which may be also relative to another timing value or relative to one or more cardiac events.

In another aspect, the disclosure provides the chamber made of an expandable material.

In another aspect, the disclosure provides the system including the processing circuitry being further configured to determine an amount of fluid in the chamber and control the pump speed relative to the amount of fluid in the chamber.

In another aspect, the disclosure provides the system including the chamber including a proximal portion and a distal portion opposite the proximal portion, the proximal portion having the housing coupled thereto and the distal portion including the valve, and the pump speed and the pump current define a one-way fluid flow path through the chamber from the proximal portion of the chamber to the distal portion of the chamber.

In another aspect, the disclosure provides the system including the processing circuitry being further configured to execute a plurality of phases including a cycle start and a filling phase, and the processing circuitry is further configured to increase the pump speed and increase the pump current relative to the cycle start during the filling phase, and wherein the pump speed and the pump current define a target pressure value in the chamber.

In another aspect, the disclosure provides the target pressure value in the chamber being associated with a transition of a cardiac cycle of the patient from a diastole phase to a systole phase.

In another aspect, the disclosure provides the filling phase being associated with a diastolic phase of a cardiac cycle of a patient when the blood pump is implanted in the patient and the ejection phase is associated with a systolic phase of the cardiac cycle.

In another aspect, the disclosure provides the system including a fixation device coupled to the housing and including a heartbeat sensor coupled thereto; an intracorporal driveline extending from the housing and being in communication with the rotor; and a power source coupled to the intracorporal driveline.

In another aspect, the disclosure provides the system including the processing circuitry being further configured to detect heartbeat data from the heartbeat sensor and transition the valve from the open position to the closed position based on the heartbeat data.

In one aspect, the present disclosure provides an intra-ventricular pulsatile assist system including an intra-ventricular blood pump including a chamber defining a fluid cavity and having a distal portion and a proximal portion opposite the distal portion, the proximal portion and the distal portion defining an axis extending therebetween and the distal portion defining an outlet, and the chamber being of an expandable material; a valve at the distal portion of the chamber, the valve including a closed position in which the outlet is sealed and an open position in which the outlet is unsealed; a housing coupled to the chamber and having a rotor and a motor therein; a fixation device coupled to the housing and including a heartbeat sensor coupled thereto; an intracorporal driveline extending from the housing and being in communication with the rotor; and a power source coupled to the intracorporal driveline; and a control circuit including a processor in communication with the blood pump, the processor having processing circuitry configured to control a pump speed of the rotor and a pump current of the motor, the pump speed and the pump current defining a pressure value in the chamber and a one-way fluid flow path through the chamber from the proximal portion of the chamber to the distal portion of the chamber; determine the defined pressure value in the chamber; compare the determined pressure value in the chamber to a predetermined threshold value, and the predetermined threshold value is one of a group consisting of a target pressure value for the chamber and a predetermined time interval associated with a cardiac cycle of a patient when the blood pump is implanted in the patient; and transition the valve between the closed position and the open position when the determined pressure value deviates from the predetermined threshold value.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an intra-ventricular pulsatile assist system that illustrates the system including a blood pump implanted in a patient, an intracorporal driveline extending from the blood pump, a power source coupled to the driveline, and a charging device positioned external to the patient for charging the power source;

FIG. 2 is a front view of the blood pump of FIG. 1 that illustrates the blood pump including a chamber defining a fluid outlet, a valve coupled to the chamber, the valve having a closed position in which the fluid outlet is sealed, and the system including a housing coupled to the chamber and a fixation device coupled to the housing;

FIG. 3 is a front partial cutaway view of the blood pump of FIG. 1 that illustrates the valve in an open position in which the fluid outlet is unsealed and the system including a rotor and a motor within the housing;

FIG. 4 is a right-side partial cutaway view of the fixation device of FIG. 2 that illustrates the fixation device including one or more electrodes coupled thereto;

FIG. 5 is a top view of the fixation device of FIG. 2 that illustrates one configuration of the electrodes;

FIG. 6 is a block diagram that illustrates the power source of FIG. 1;

FIG. 7 is a block diagram that illustrates the charging device of FIG. 1;

FIG. 8 is a block diagram that illustrates the system including a control system for controlling the rotor and the motor of FIG. 3;

FIG. 9 is a graph that illustrates an amount of fluid flow into and out of the chamber of FIG. 1, an aortic pressure of the patient, a number of phases executed by the system, and the status of the valve relative to the phases;

FIG. 10 is a graph that illustrates a pump speed, a pump current, the phases of FIG. 10, and the status of the valve relative to the phases;

FIG. 11 is a graph that illustrates the measurements of FIGS. 10 and 11 and a volume of fluid in the chamber relative to the phases;

FIG. 12 is a front partial cutaway view of the blood pump of FIG. 1 that illustrates the system 10 in a filling phase in which the amount of fluid into the chamber increases and the valve is in the closed position;

FIG. 13 is a front partial cutaway view of the blood pump of FIG. 1 that illustrates the system 10 in an ejection phase in which the fluid in the chamber is ejected from the chamber through the fluid outlet;

FIG. 14 is a flow diagram that illustrates a method of transitioning the valve of from the closed position of FIG. 2 to the open position of FIG. 3;

FIG. 15 is a flow chart depicting an overview of the system of FIG. 1 and exemplary instructions which may be executed by processing circuitry of the system in timing and performing the phases of the system;

FIG. 16 is a flow chart of the system of FIG. 1 including exemplary instructions for controlling the pump speed during the filling phase of FIG. 13 in which the amount of fluid into the chamber increases and the valve is in the closed position;

FIG. 17 is a flow chart of the system of FIG. 1 including exemplary instructions for controlling the pump speed during the filling phase in which the pressure value in the chamber is increased toward a target pressure value for the chamber; and

FIG. 18 is a flow chart of the system of FIG. 1 including exemplary instructions for controlling the pump speed during the ejection phase in which the fluid in the chamber is ejected from the chamber through the fluid outlet.

### DETAILED DESCRIPTION

Before describing in detail exemplary embodiments, it is noted that the configurations reside primarily in combinations of device and system components, as well as method steps related to providing pumping assistance to the heart in a pulsatile manner. Accordingly, the device, system, and method components have been represented where appropriate by conventional symbols in the drawings, showing only those specific details that are pertinent to understanding the configurations of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate, and modifications and variations are possible of achieving the electrical and data communication.

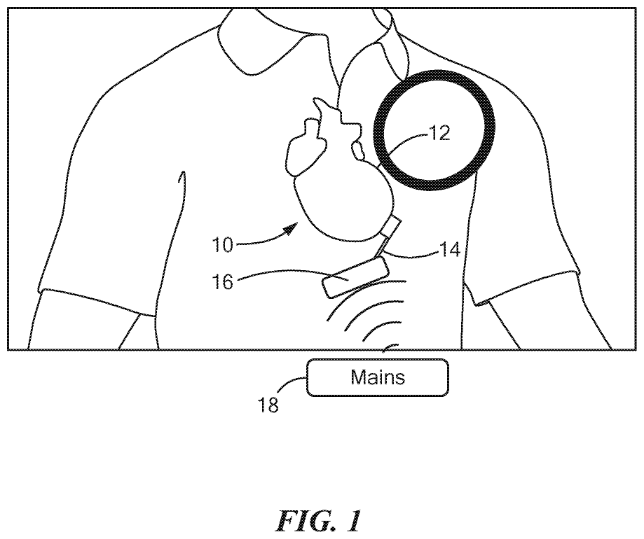

Referring now to the drawings in which like reference designators refer to like elements there is shown in FIG. 1 an intra-ventricular pulsatile assist system 10 including an intra-ventricular blood pump 12 implanted within a heart of a patient, for example, within the left ventricle. The blood pump 12 may also be implanted within the right ventricle of the patient as the left ventricle of FIG. 1 is not intended to be limiting. In one example, the pulsatile flow of the system 10 is configured to provide pumping assistance to the heart, thus providing the opportunity to promote intrinsic movement of the heart and strength which may be beneficial for patients who are candidates for weaning off of the blood pump 12. In another example, the pulsatile flow of the system 10 is configured to provide permanent circulatory support to a patient.

The system 10 includes an intracorporal driveline 14 extending from the blood pump 12 to a power source 16 configured to supply power to the blood pump 12. In one example, an external charging device 18 is used to charge the power source 16, however, such example is not intended to be limiting. The system 10 is configured to be minimally invasive upon implantation and consume minimal power through, for example, transcutaneous transfer between the charging device 18 and the power source 16.

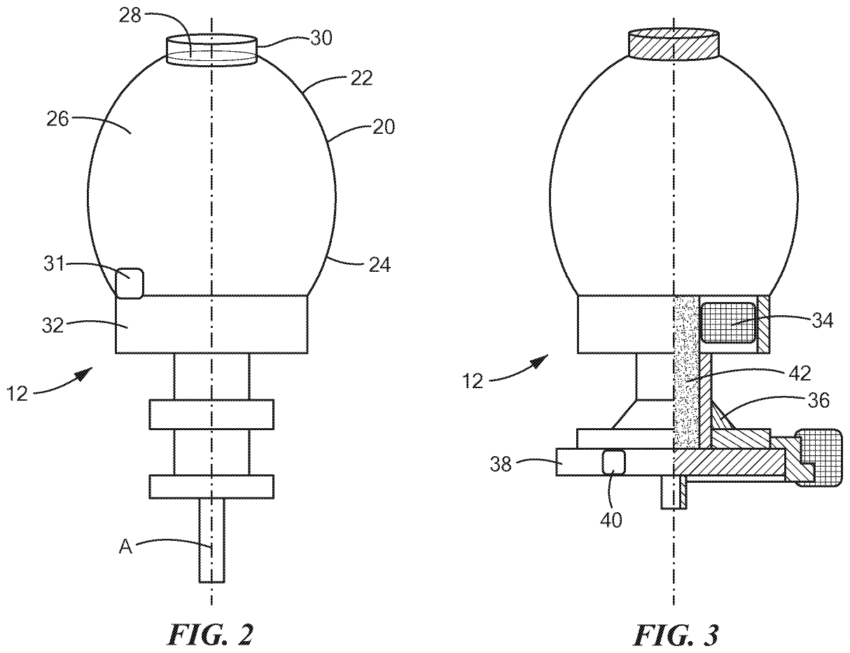

FIG. 2 is a front view of the blood pump 12 including a chamber 20 having a distal portion 22 and a proximal portion 24 opposite the distal portion 22 which define an axis “A” extending therethrough. The chamber 20 defines a fluid cavity 26 with the distal portion 22 defining an outlet 28 in communication with the fluid cavity 26. The chamber 20 is made of an expandable material configured to expand as fluid, e.g., blood, enters and pressure builds within the chamber 20. FIG. 2 depicts the chamber 20 as an elastic balloon, however, the chamber 20 may also be a referred to as a flexible balloon, sack, or another expandable device configured in various geometrical shapes configured to promote unidirectional flow of the fluid within the chamber 20.

The material of the chamber 20 may be knitted fibers, e.g., a knitted fabric, reinforced with various types of material to promote fluid flow within the chamber 20. In one example, the chamber 20 is configured to promote fluid flow from the proximal portion 24 to the distal portion 22 of the chamber 20 in a relatively continuous manner to prevent clotting. Further, the material is biocompatible to prevent emboli or thrombi formation or other immune responses. One or more measurement indicators may be provided on or in the material (not shown) to measure strain, stress, volume, expansion, and/or a pressure within the chamber 20 used to determine the timing of an ejection of the fluid.

The blood pump 12 includes a valve 30 at the distal portion 22 of the chamber 20. FIG. 2 depicts the valve 30 including a closed position in which the outlet 28 is sealed and FIG. 3 depicts the valve 30 including an open position in which the outlet 28 is unsealed. The valve 30 is configured to retain the fluid and corresponding pressure within the chamber 30 until the pressure deviates from, such as exceeds, a predetermined threshold value which may be expressed as an adjustable range to vary the level of support provided to the heart, for example, as the heart strengthens. The blood pump 12 may include a sensor 31 for determining the amount of fluid and/or pressure within the chamber 20. The open position of the valve 30 allows fluid to be ejected through the outlet 28 for transfer to the aorta or pulmonary valve of the patient. Although FIG. 2 depicts the valve 30 as a flexible ring, in other examples, the valve 30 may be a bi-leaflet valve, a pattern of knit fibers or loops, such as magnetic or metallic fibers interwoven with the chamber 20, a coating, or the chamber 20 may define the valve 30 as an aperture, the diameter of which may be selectively sized to open and seal the chamber 20.

FIG. 2 depicts the chamber 20 including a housing 32 coupled to the proximal portion 24 thereof for housing, as shown in FIG. 3, a rotor 34 and a motor 36 configured to provide a pump speed and a pump current, respectively, to operate the blood pump 12. The blood pump 12 is not limited to use of the rotor 34 to impel fluid as other devices suitable for impelling fluid may be used. A single housing 32 or separate housings may be used to house or cover the rotor 34 and the motor 36. The system 10 includes a fixation device 38, e.g., a sewing ring, configured to be in contact with the patient's myocardium to position the blood pump 12 within the heart upon implantation. FIG. 3 also depicts the housing 32 including a heartbeat sensor 40 for sensing data associated with the patient's heartbeat. The data may be transferred to a remote system through a transmitter and receiver (not shown) and used by the system 10 to time phases thereof with the phases of the cardiac cycle. In one example, the heartbeat sensor 40 is an electrocardiography (ECG) sensor. In another example, the sensor is a vibration or accelerometer sensor with one or more axis, however other sensors may be used. A hub 42 extends within the housing 32 proximate the rotor 34 and the motor 36 which may couple to the intracorporal driveline (FIG. 1) to supply the pump current to the blood pump 12. If there is not enough space to house the motor 36 within the housing 32, the motor 36 may be extended through the fixation device 38 to outside of the myocardium.

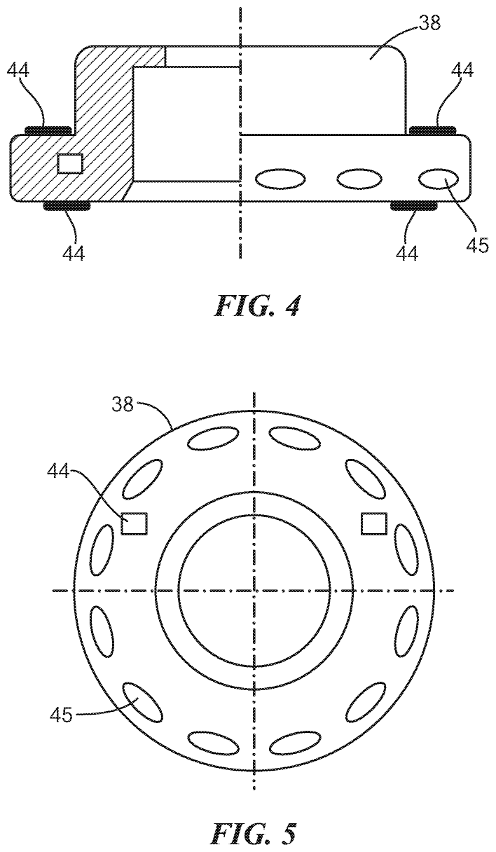

FIGS. 4 and 5 are a right-side partial cutaway view and a top view of the fixation device 38, respectively, including one or more electromyography (“EGM”) electrodes 44 configured to transfer the pump current from the driveline 14 and power source 16 to the blood pump 12. In other examples, an alternative type of electrode or various types of electrodes may be coupled to the fixation device 38 and/or an epicardial electrode may be coupled to the heart to supply power to the blood pump 12 from the power source 16. The fixation device 38 is depicted defining apertures 45 for suturing the fixation device 38 to the myocardium (i.e., apex).

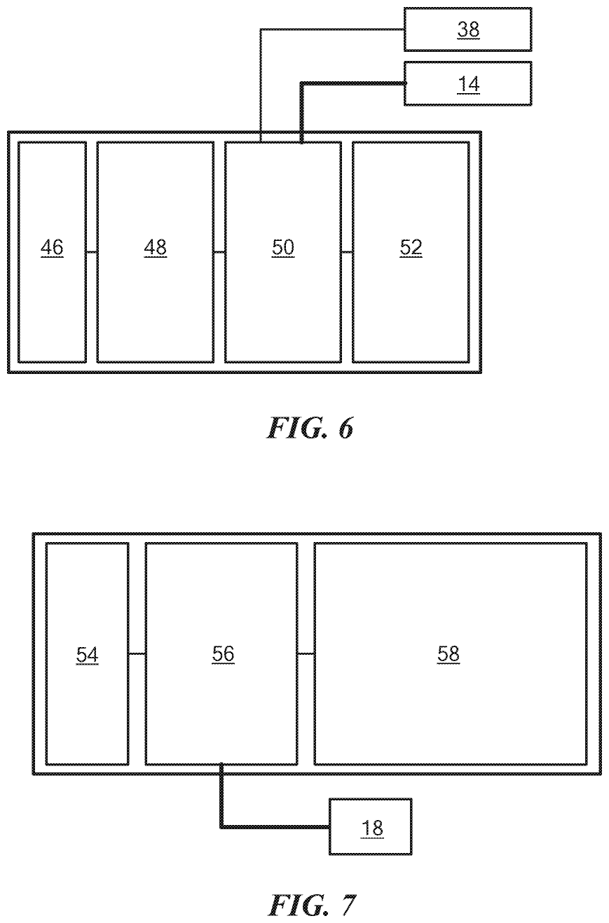

FIG. 6 is a block diagram of the power source 16 including a charging coil 46, a battery 48, an electronic hybrid board 50, wherein the electronic hybrid board 50 is coupled to the driveline 14 in communication with the electrodes 44 of the fixation device 38. In addition, the power source 16 may include an energy storage device 52 for storing energy to allow for relatively quick interval current needs of the pump as determined according to the individual patient's needs. The power source 16 may be implanted in the patient's upper abdominal area or another suitable location relative to the heart. The components of the power source 16 depicted in FIG. 6 are provided for exemplary purposes and are not intended to be limiting.

FIG. 7 is a block diagram of the charging device 18 coupled to a mains electricity for transmitting power or energy to the power source 16 (FIG. 6) through transcutaneous transfer or other transmission when the charging device 18 is external to the patient. In one configuration, the charging device 18 includes a charging coil 54, an electronic hybrid board 56, and a battery 58, however, such components are exemplary and not intended to be limiting.

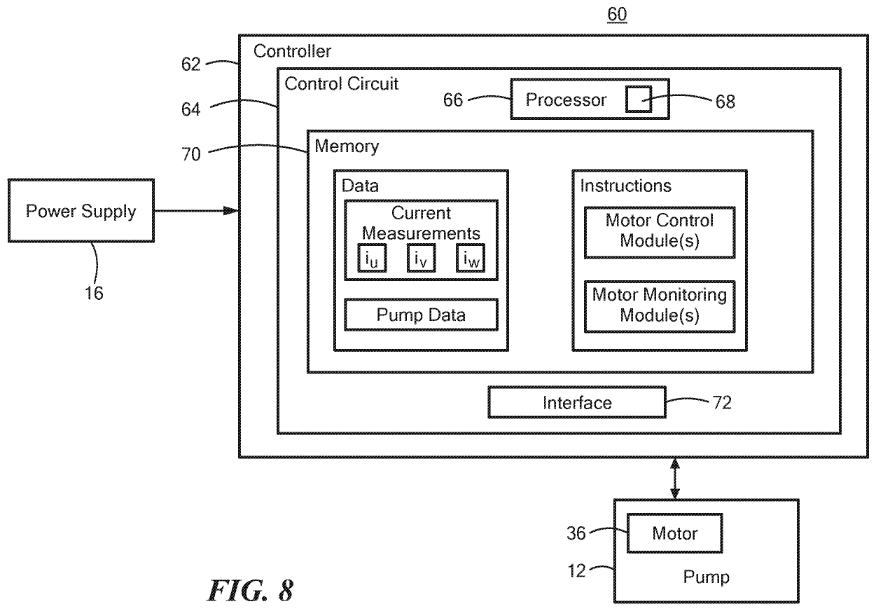

FIG. 8 is a block diagram of the system 10 including a control system 60 in communication with the blood pump 12 and the power source 16 for controlling the motor 36 and other operational components of the blood pump 12. In one example, the control system 60 includes a controller 62 having a control circuit 64 and a processor 66 including processing circuitry 68 configured to perform one or more operations of the blood pump 10. The system 60 may also include a memory 70 and an interface 72, the memory 70 being configured to store information accessible by the processor 66, including instructions executable by the processing circuitry 68 and/or data that may be retrieved, manipulated or stored by the processor 66. Such instructions and/or data include that which is used to control the pump speed and the pump current, determine a pressure value within the chamber 30 e.g., defined by the pump speed and the pump current, and transition the valve 30 between the open and closed positions. Reference to the system 10 is intended to include reference to the control system 60 for pump operation.

The system 10 is configured to carry out phases or a cycle using the blood pump 12 that correlate to the diastole and systole phases of the patient's cardiac cycle. The phases include a cycle start and a filling phase in which the chamber 20 fills with a fluid, e.g., blood, an ejection in which the valve 30 opens, an ejection phase in which the fluid is ejected from the chamber 20, and an ejection-end phase occurring after the ejection phase. The system 10 is configured to time the ejection as the cardiac cycle transitions from the diastole phase to the systole phase. As such, when the blood pump 12 is in the left ventricle, the fluid is ejected toward the aorta through the open aortic valve and when the blood pump 12 is in the right ventricle, the fluid is ejected through the open pulmonary valve into the pulmonary vein to promote perfusion.

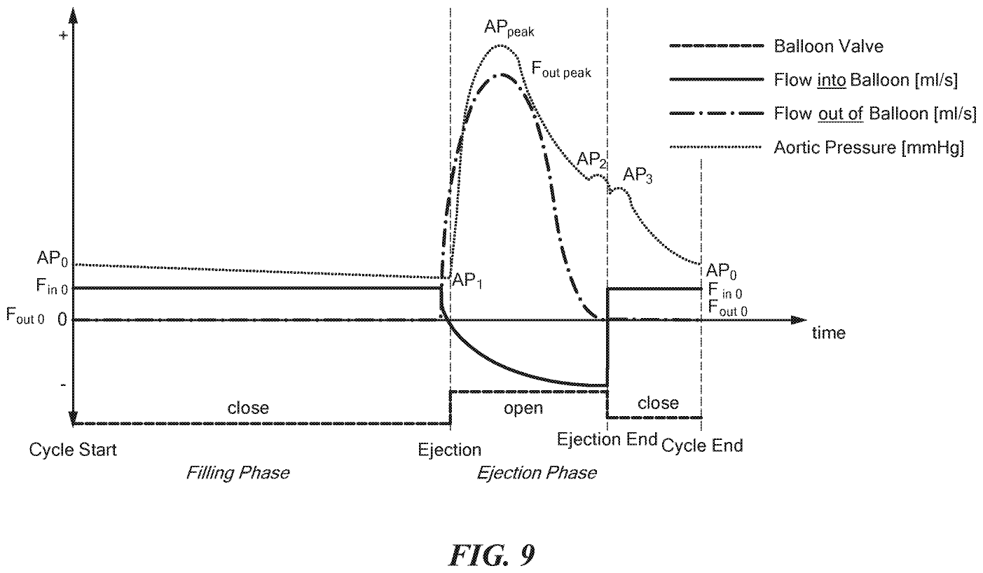

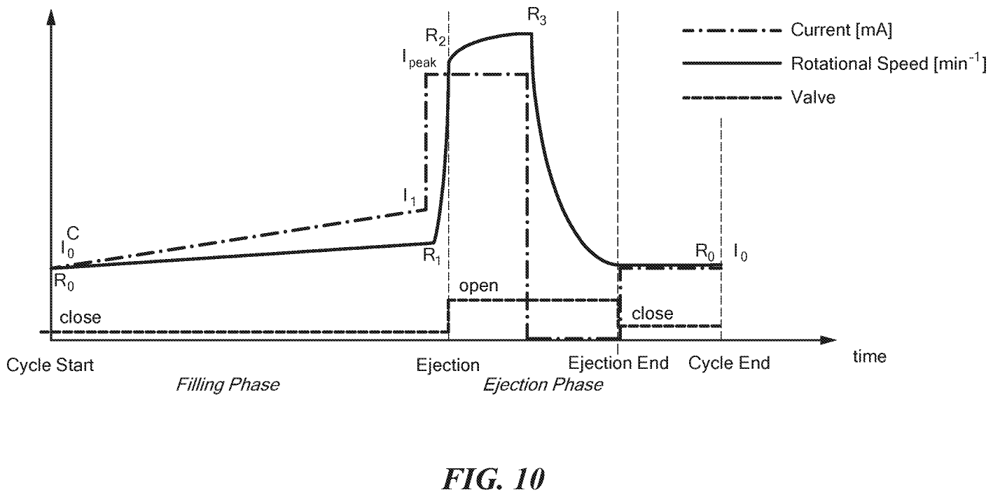

FIGS. 9-11 are graphs depicting waveforms indicating measurements associated with the phases. FIG. 9 is a graph depicting the waveforms representing the fluid flow into the chamber 30 designated as “F in,” the fluid flow out of the chamber 30 designated as “F out,” and an aortic pressure of the patient designated as “AP”. The waveforms are plotted over time and relative to the phases labeled along the Y axis. The Y axis also includes status indicators depicting the status of the valve 30, i.e., the open position or the closed position, relative to the phases and waveforms.

During the cycle start, the cardiac cycle is in the diastole phase, i.e., the heart is relaxed. The chamber 20 contains a minimal volume of fluid and thus a minimal amount of pressure as the amount of pressure in the chamber 20 is correlated to the amount of fluid. The pump speed and the pump current are at a minimum for the phases as the blood pump 12 is working against the minimal pressure or resistance to transfer fluid into the chamber 20. The system 10 is configured to promote fluid entering the chamber 20 at a relatively steady rate, suction is absent, and the valve 30 is in the closed position.

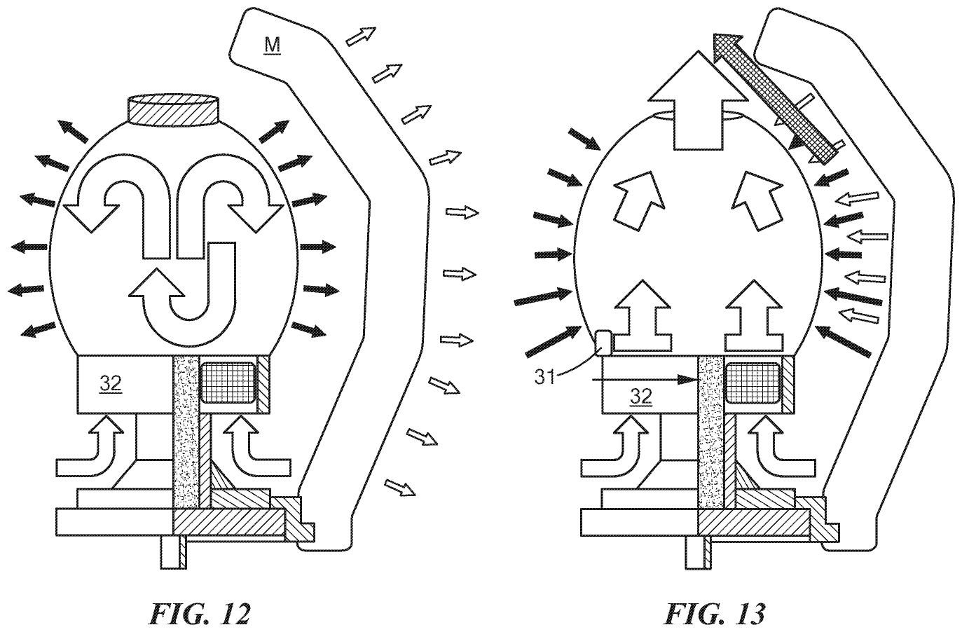

FIGS. 9-11 may be viewed with FIG. 12 which depicts a front partial cutaway view of the blood pump 12 including the system 10 in the filling phase which includes the chamber 20 beginning to fill with fluid, e.g., blood, and the valve 30 in the closed position. The cardiac cycle of the patient remains in diastole. The fluid enters the blood pump 12 through an inlet of the housing 32 (not shown) and the chamber 20 is configured to expand as the fluid enters due to the increasing pressure. FIG. 12 also depicts the blood pump 12 upon implantation and proximate the patient's myocardium “M” which contracts in an effort to propel the fluid toward the outlet 28 and the aorta or pulmonary vein of the patient.

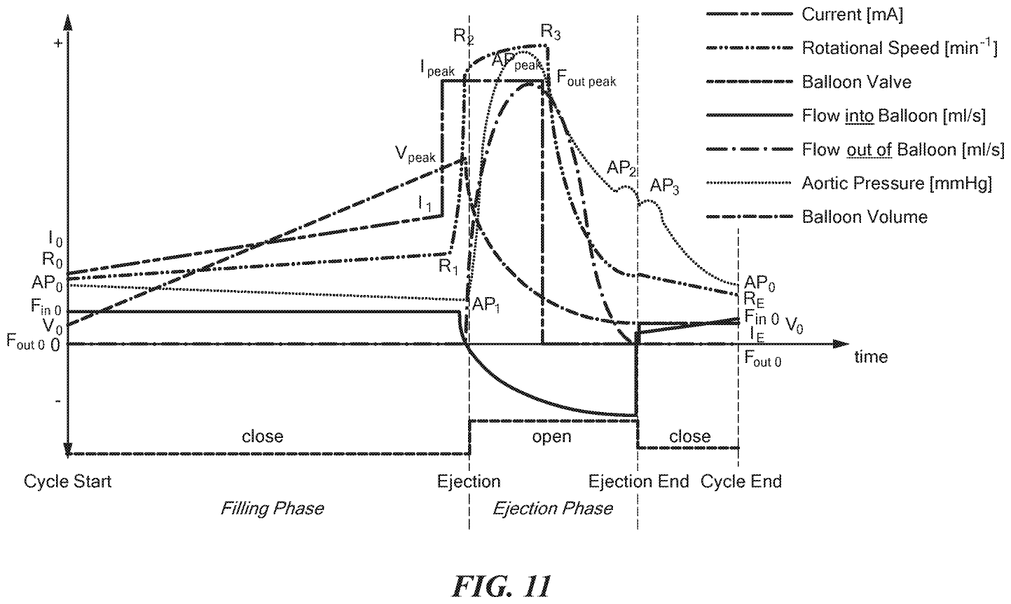

The processing circuitry 68 is configured to determine the amount of fluid in the chamber 20, for example using the sensor 31 (FIG. 2) and control the pump speed relative to the determined amount of fluid. In addition, because the pressure value in the chamber corresponds to the amount of fluid in the chamber 20, the pump speed may also be controlled relative to the pressure value. For example, as shown in FIG. 11, as the fluid volume and pressure in the chamber 20 increase, the processing circuitry 68 is configured to increase the pump speed relative to the cycle start to an increased speed. The pump speed increase may be constant throughout the filling phase or incremental to promote a relatively constant flow of the fluid flow into the chamber 20 against the rising pressure and to act as a valve to prevent the fluid flow from traveling out of the chamber 20 away from the outlet 28. As such, the pressure value in the chamber 20 is below the predetermined pressure value during the filling phase.

Referring still to FIG. 11, to meet the power or energy demand resulting from the increase in the pump speed, the processing circuitry 68 is configured to increase the pump current relative to the cycle start to an increased current during the filling phase. In one example, the system 10 is configured to transition from the filling phase to ejection by generating a target pressure value in the chamber 20 through increasing the pump speed relatively rapidly in comparison to the other phases and increasing the pump current to a maximum current for the phases. As such, the pump speed and the pump current can be said to define the pressure value in the chamber 20. The pump speed increase is depicted in FIG. 11 from R1 to R2 and the increase in the pump current is depicted from I1 to Ipeak. The target pressure value in the chamber may be determined using one or more pressure values associated with the patient and/or the system 10. In another example, the processing circuitry 68 is configured to transition the system 10 from the filling phase to ejection when the cardiac cycle transitions from diastole to systole as determined using the data from the heartbeat sensor 40. Ejection occurs when the pump speed is at R2, the pump current is at Ipeak, and as the cardiac cycle transitions from diastole to systole.

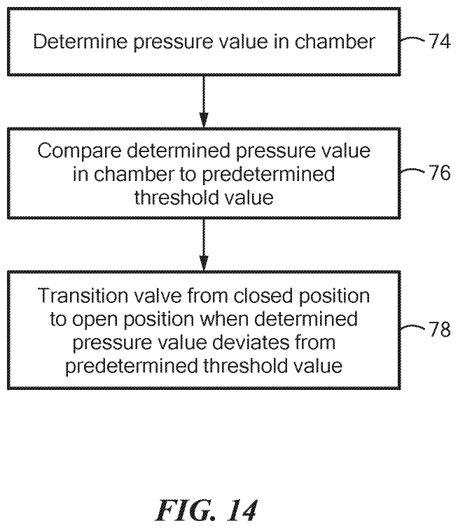

The ejection phase follows the ejection and includes, as shown in FIG. 13, the fluid being expelled from the fluid outlet 28 of the blood pump 12. FIGS. 9-11 and FIG. 13 may be viewed with FIG. 14 depicting a flow chart of method steps for timing and executing the ejection and the ejection phase with the cardiac cycle. In one configuration, the method begins at step 74 including the processing circuitry 68 configured to determine the pressure value in the chamber 30 defined by the pump speed and the pump current. At step 76, the processing circuitry 68 is configured to compare the determined pressure value in the chamber 30 to a predetermined threshold value. At step 78, the processing circuitry 68 is configured to communicate with the blood pump 12 to transition the valve 30 between the closed position (FIG. 12) and the open position (FIG. 13) when the determined pressure value deviates from, such as exceeds, the predetermined threshold value. In one example, the term “deviates” includes the determined pressure value exceeding the predetermined threshold value.

In one configuration, the predetermined threshold value is a target pressure value for the chamber 20. The system 10 may be configured to determine the target pressure value in the chamber 20 by measuring the amount of the pump current used to maintain the pump speed relatively constant. In another example, or in addition to measuring the pump current, the system 10 may determine the target pressure value in the chamber 20 using, as shown in FIG. 13, the sensor 31 in communication with the chamber 20. In another configuration, the predetermined threshold value is a predetermined time interval associated with the cardiac cycle of the patient that correlates to the predetermined time period when the cardiac cycle transitions from diastole to systole based on the individual patient's cardiac cycle. The heartbeat sensor 40 may be used to determine the timing of the cardiac cycle. The predetermined time interval associated with the cardiac cycle of the patient may be determined using additional or alternative timing values associated with the cardiac cycle, the patient, the system 10 and/or relative to one or more cardiac events. The target pressure value may be expressed as the adjustable range determined relative to a condition of the heart of the patient, such as on scale measuring a strength of the heart specific to the patient. The adjustable range may be determined by a physician or other treatment provider and programmed within the system 10 accordingly.

The system 10 may transition the valve 30 between the open and closed positions using the processing circuitry 68 in communication with a knob, button, control circuit, or other transitioning device. More specifically, mechanical or electrical impulses may communicate with the valve 30 to assist in the transition of the valve 30 from the open position to the closed position. In other examples, the target pressure value that triggers the opening of the valve 30 is generated as a pressure peak within the chamber 20. The pressure peak may be initiated using heartbeat data, i.e., cardiac data, from the heartbeat sensors 40, a time interval programmed in the controller 62, and/or a flow value, elongation, strain or a pressure value from the sensors 31 or alternative sensors located in or around the heart or the system 10. The pressure peak may be associated with the maximal expected systolic pressure for the patient. In another example, when the patient's heart strengthens such that the patient produces a higher systolic pressure relative to a previous pressure, the myocardium may squeeze the chamber 30 to increase the pressure value in the chamber 30, thereby causing the valve 20 to open. Upon the opening of the valve 20, the chamber 30 is washed by the rotor 34.

In another example, with reference to FIGS. 4 and 5, the heartbeat sensors 40 on the fixation device 38 are configured to provide ventricular stimulation to force the system 10 into the ejection following the filling phase in conjunction with the cardiac cycle. In one example, the intracorporal driveline 14 and the controller 62 are used to implement the ventricular stimulation, although such example is not intended to be limiting. Further, an accelerometer (not shown) is configured to sense the physical activity of the patient and adapt the stimulation frequency relative to such physical activity. The ventricular stimulation may be beneficial when the signals from sensors on or in the chamber 20 are relatively weak or absent and/or when the intrinsic heart rate is too slow and thus insufficient for determining the timing of the ejection. In addition, the ventricular stimulation may be beneficial when the heart-minute-volume would benefit from an increase in the heart rate to increase the heart-minute-volume to a select level. As such, the system 10 not only incorporates cardiac sensing feasibility to time the ejection, but also provides a cardiac stimulation function to perform the ejection.

Referring again to FIG. 13, depicting a front partial cutaway view of the blood pump 12, the pump speed and the pump current define a one-way fluid flow path along the axis A of the chamber 20 from the proximal portion 24 to the distal portion 22 of the chamber 20 and extending through the outlet 28. The chamber 20 contracts at the time of the heart's contraction during the ejection phase. The Bernoulli's principle and effect of the contracting chamber 20 promote the ejection of the fluid between the chamber 20 and a wall of the myocardium toward the outlet 28 and the aorta or pulmonary vein. In other words, the relatively low amount of pressure between the chamber 20 and the myocardium during the ejection phase as compared to the filling phase promotes the contraction of the myocardium.

As shown in FIG. 11, the pump speed remains relatively high at R2 to R3 after the opening of the valve 30 for a relatively short period to prevent fluid regurgitation through the blood pump 12 and to drain the apex from the fluid when the fluid is blood. The system 10 may be configured to reduce the pump current relative to Ipeak as a result of the relatively rapid acceleration of the pump speed to reach ejection which results in inertia and continued rotation. The amount of fluid remaining in the chamber 20 is minimal as the ejection phase ends.

The ejection-end phase follows the ejection phase and includes the valve 30 transitioning from the open position to the closed position. When the aortic pressure of the patient is as large as the pressure value of the chamber 20 at the ejection, the aortic valve of the patient will begin to close. The fluid flow into the chamber 20 is minimal or nonexistent, depending on the amount of fluid volume remaining in the ventricle of the patient, and the volume of fluid in the chamber 20 is minimal. The system 10 is configured to decrease the pump speed from R3 to the standard set speed in a direction toward R0. In addition, the system 10 is configured to control the pump current relative to the pump speed and thus the pump current may be decreased with the decrease in the pump speed to save energy.

The phases of the system 10 or cycle ends after the ejection-end phase during which the valve 30 is in the closed position and the pump speed is relatively low compared to the filling phase. The pump speed is increased during the cycle start when the chamber 20 begins to fill with the fluid. In one configuration, the system 10 is configured to communicate with the heartbeat sensor 40 to determine a timing of the completion of the ejection-end phase and thus the cycle end and the subsequent cycle start.

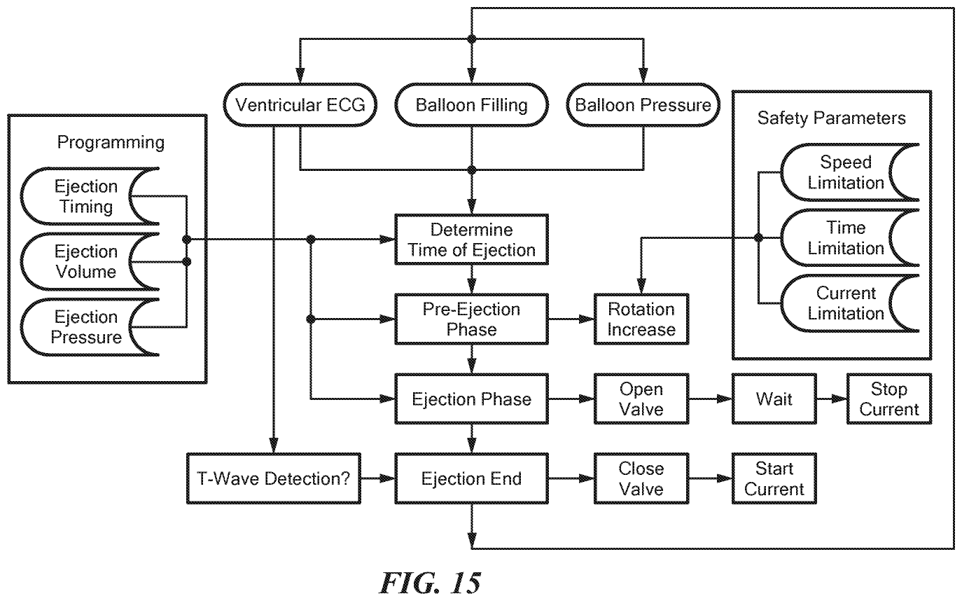

FIG. 15 is a flow chart depicting an overview of the system 10 and exemplary instructions which may be executed by the processing circuitry 68 in timing and performing the phases or cycle of the system 10. In one configuration, the instructions include programming the predetermined threshold value which may include an ejection timing, i.e., the predetermined time interval, an ejection volume, and an ejection pressure, i.e., the target pressure value of the chamber 20. In addition, the instructions may include value inputs associated with the ECG, the filling of the chamber 20, and/or the pressure value within the chamber 20, as well as safety parameters for use in determining the timing of the phases.

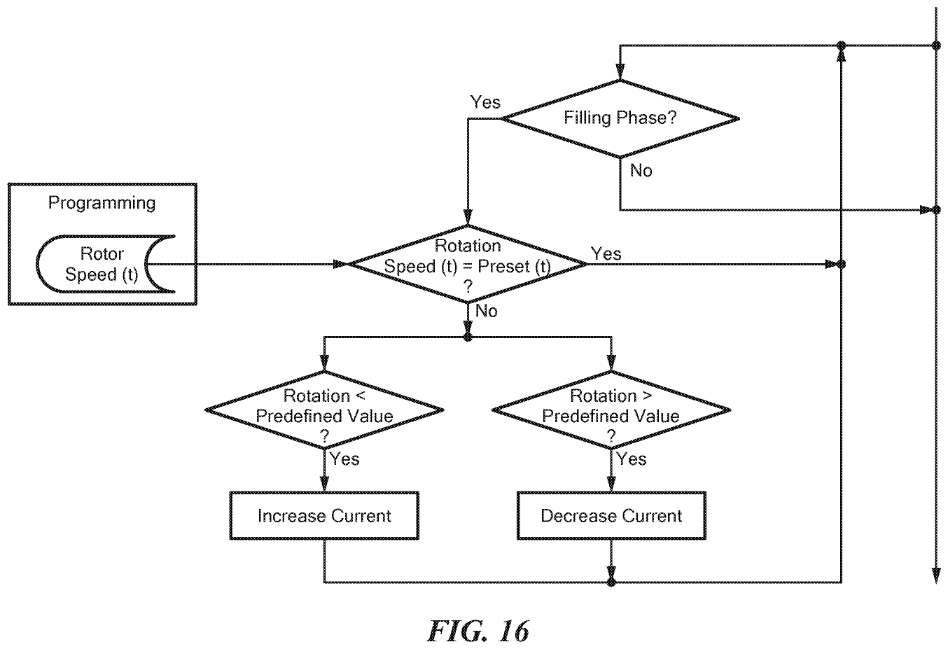

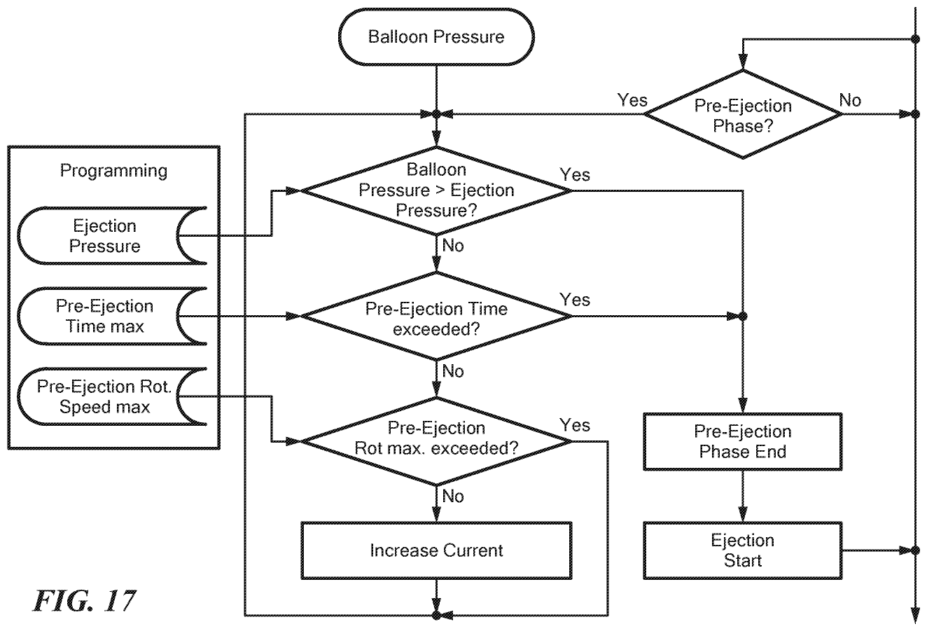

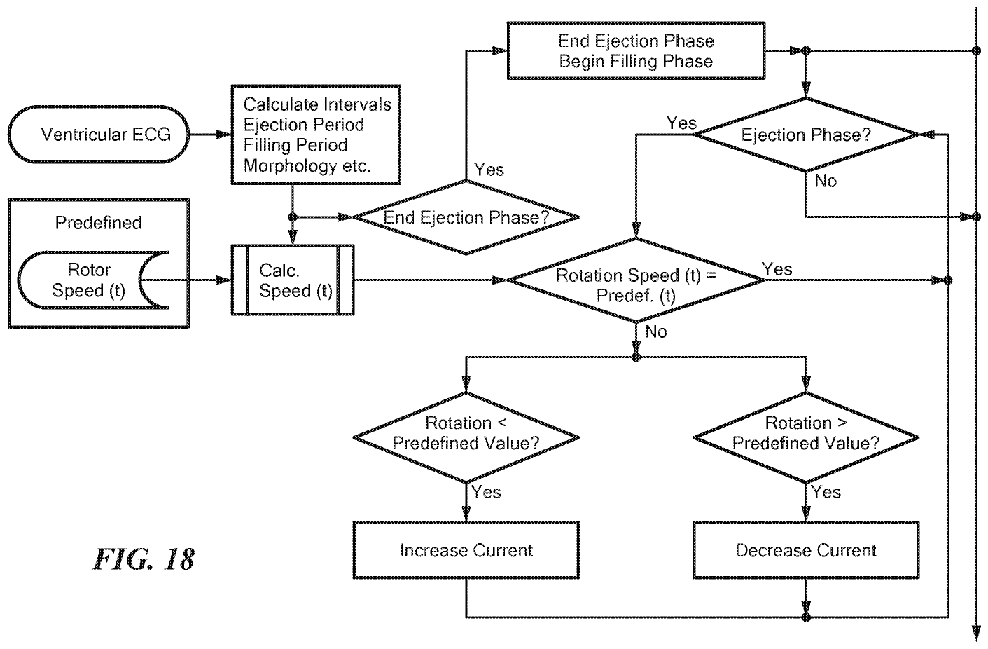

FIGS. 16-18 depict flow charts of the system 10 and exemplary instructions for controlling the pump speed during the filling phase and the ejection phase. Such instructions are provided for exemplary purposes only and are not intended to be limiting. Further, the steps may be carried out in a different order and additional steps may be added or others omitted.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims

What is claimed is:

1. An intra-ventricular pulsatile assist system comprising:
an intra-ventricular blood pump including:
a chamber having a distal portion and a proximal portion opposite the distal portion, the proximal portion and the distal portion defining an axis extending therebetween and the distal portion defining an outlet;
a valve at the distal portion of the chamber, the valve including a closed position in which the outlet is sealed and an open position in which the outlet is unsealed; and
a control circuit including a processor in communication with the blood pump, the processor having processing circuitry configured to:
determine a pressure value in the chamber; and
transition the valve between the closed position and the open position when the pressure value in the chamber deviates from a predetermined threshold value.

2. The system of claim 1, wherein the predetermined threshold value is a target pressure value for the chamber associated with a condition of a heart of a patient, and the chamber is of an expandable material.

3. The system of claim 1, wherein the predetermined threshold value is a predetermined time interval associated with a cardiac cycle of a patient having the blood pump implanted in the patient.

4. The system of claim 1, wherein the processing circuitry is further configured to: execute a plurality of phases, control a pump speed of the blood pump relative to the plurality of phases, and control a pump current of the blood pump relative to the plurality of phases, and wherein the pump speed and the pump current define the pressure value in the chamber.

5. The system of claim 4, wherein the processing circuitry is further configured to increase the pump speed and increase the pump current relative to one of the group consisting of an aortic pressure and a pulmonary pressure of the patient.

6. The system of claim 5, wherein the pump speed and the pump current define a one-way fluid flow path along the axis of the chamber from the proximal portion of the chamber to the distal portion of the chamber.

7. The system of claim 6, wherein the plurality of phases include a cycle start and a filling phase, and the processing circuitry is further configured to increase the pump speed and increase the pump current relative to the cycle start during the filling phase, and wherein the pump speed and the pump current define a target pressure value in the chamber timed in association with a transition of a cardiac cycle of the patient from a diastole phase to a systole phase.

8. The system of claim 1, further comprising:
a housing coupled to the proximal end of the chamber and including a rotor disposed therein;
a fixation device coupled to the housing and having a heartbeat sensor coupled thereto; an intracorporal driveline extending from the housing and being in communication with the rotor; and
a power source coupled to the intracorporal driveline.

9. An intra-ventricular pulsatile assist system comprising:
an intra-ventricular blood pump including:
a chamber defining a fluid cavity and an outlet in fluid communication with the fluid cavity;
a valve coupled to the chamber and having a closed position in which the outlet is sealed and an open position in which the outlet is unsealed; and
a housing couple to the chamber and having a rotor and a motor therein; and a control circuit including a processor in communication with the blood pump, the processor having processing circuitry configured to:
control a pump speed of the rotor and a pump current of the motor, the pump speed and the pump current defining a pressure value in the chamber;
determine the defined pressure value in the chamber;
compare the determined pressure value in the chamber to a predetermined threshold value associated with one of a group consisting of an aortic pressure value and a pulmonary pressure value of a patient having the blood pump implanted in the patient; and
transition the valve between the closed position and the open position when the determined pressure value deviates from the predetermined threshold value.

10. The system of claim 9, wherein the predetermined threshold value is a target pressure value for the chamber.

11. The system of claim 9, wherein the predetermined threshold value is a predetermined time interval associated with a cardiac cycle of the patient.

12. The system of claim 9, wherein the chamber is made of an expandable material.

13. The system of claim 9, wherein the processing circuitry is further configured to determine an amount of fluid in the chamber and control the pump speed relative to the amount of fluid in the chamber.

14. The system of claim 9, wherein the chamber includes a proximal portion and a distal portion opposite the proximal portion, the proximal portion having the housing coupled thereto and the distal portion including the valve, and the pump speed and the pump current define a one-way fluid flow path through the chamber from the proximal portion of the chamber to the distal portion of the chamber.

15. The system of claim 9, wherein the wherein the processing circuitry is further configured to execute a plurality of phases including a cycle start and a filling phase, and the processing circuitry is further configured to increase the pump speed and increase the pump current relative to the cycle start during the filling phase, and wherein the pump speed and the pump current define a target pressure value in the chamber.

16. The system of claim 15, wherein target pressure value in the chamber is associated with a transition of a cardiac cycle of the patient from a diastole phase to a systole phase.

17. The system of claim 16, wherein the filling phase is associated with a diastolic phase of a cardiac cycle of a patient when the blood pump is implanted in the patient and the ejection phase is associated with a systolic phase of the cardiac cycle.

18. The system of claim 9, further comprising:
a fixation device coupled to the housing and including a heartbeat sensor coupled thereto;
an intracorporal driveline extending from the housing and being in communication with the rotor; and
a power source coupled to the intracorporal driveline.

19. The system of claim 18, wherein the processing circuitry is further configured to detect heartbeat data from the heartbeat sensor and transition the valve from the open position to the closed position based on the heartbeat data.

20. An intra-ventricular pulsatile assist system comprising:
an intra-ventricular blood pump including:
a chamber defining a fluid cavity and having a distal portion and a proximal portion opposite the distal portion, the proximal portion and the distal portion defining an axis extending therebetween and the distal portion defining an outlet, and the chamber being of an expandable material;
a valve at the distal portion of the chamber, the valve including a closed position in which the outlet is sealed and an open position in which the outlet is unsealed;
a housing coupled to the chamber and having a rotor and a motor therein; a fixation device coupled to the housing and including a heartbeat sensor coupled thereto;
an intracorporal driveline extending from the housing and being in communication with the rotor; and
a power source coupled to the intracorporal driveline; and
a control circuit including a processor in communication with the blood pump, the processor having processing circuitry configured to:
control a pump speed of the rotor and a pump current of the motor, the pump speed and the pump current defining a pressure value in the chamber and a one-way fluid flow path through the chamber from the proximal portion of the chamber to the distal portion of the chamber;
determine the defined pressure value in the chamber;
compare the determined pressure value in the chamber to a predetermined threshold value, and the predetermined threshold value is one of a group consisting of a target pressure value for the chamber and a predetermined time interval associated with a cardiac cycle of a patient when the blood pump is implanted in the patient; and
transition the valve between the closed position and the open position when the determined pressure value deviates from the predetermined threshold value.
